(19) United States
(12) Patent Application Publication
(10) Pub. No.: US 2026/0106027 A1
(43) Pub. Date: Apr. 16, 2026

(54) SUGGESTION SYSTEM

(71) Applicant: TOPCON CORPORATION, Tokyo (JP)

(72) Inventor: Noriyasu TAKEDA, Tokyo (JP)

(73) Assignee: TOPCON CORPORATION, Tokyo (JP)

(21) Appl. No.: 19/311,004

(22) Filed: Aug. 27, 2025

(30) Foreign Application Priority Data
Oct. 11, 2024 (JP) 2024-179263
Jul. 18, 2025 (JP) 2025-121367

(51) Int. Cl. G16H 50/20; A61B 3/00; G06T 7/00

(52) U.S. Cl. CPC G16H 50/20; A61B 3/0025; G06T 7/0014; G06T 2207/10101; G06T 2207/20081; G06T 2207/30041

(57) ABSTRACT

A suggestion system includes a terminal device installed in a hospital and a trained model connected to the terminal device and configured to assist in determining disease discovery. The terminal device includes a processor configured to: receive at least numerical data obtained by ophthalmic diagnosis and image data that is a result of image diagnosis of an eyeball; analyze a disease contained in the image data from similarity between a feature of the image data and a feature of an existing ophthalmic image sample using the trained model; and determine and suggest an optimum treatment for a patient based on the numerical data and an analysis result of the image data.

### CROSS-REFERENCE TO RELATED APPLICATIONS

This application is based on and claims priority under 35 USC 119 from Japanese Patent Application No. 2024-179263 filed on Oct. 11, 2024, and Japanese Patent Application No. 2025-121367 filed on Jul. 18, 2025, the entire contents of each are incorporated herein by reference.

### BACKGROUND

### 1. Field

The present disclosure relates to a suggestion system for suggesting a selection for personalized treatment and prescription tailored to a patient.

### 2. Description of the Related Art

Currently, a concept of personalized medicine is being introduced, particularly in cancer treatment. The personalized medicine refers to treatment and prescription tailored to a constitution and a sickness type of each patient. For example, information such as data related to the constitution and the sickness of the patient and data related to genes is examined more finely, and then treatment tailored to each patient is performed.

Patent Literature 1 (JP2024-074287A) related to the personalized medicine is a device using machine learning. In a medical information processing apparatus, a first acquisition unit acquires a plurality of training samples. Each of the training samples includes a feature representing a state of a subject, a type label of an event for the subject (a medical action performed by a medical worker or an action performed by the subject), and an effect label of the event. Further, a second acquisition unit acquires a knowledge base from the plurality of training samples.

An assignment unit assigns knowledge labels (ground truth data in the machine learning) to at least a part of the plurality of training samples based on the knowledge base. Then, a training unit trains a model that infers an effect for each type of event based on the training samples to which the knowledge labels are assigned. That is, at least a part of the training samples each includes the feature, the type label, the effect label, and the knowledge label. Since the effect for each event type is inferred in consideration of not only the training sample but also the knowledge base, accuracy of a causal inference model can be improved with a small number of training samples. The inference is very useful for treatment of patients (see paragraphs 0006, 0066, 0067 of Patent Literature 1).

### SUMMARY

In a case of eye diseases such as glaucoma, age-related macular degeneration (AMD), and diabetic retinopathy (DR), the same medicine is often prescribed for patients with the same sickness and symptom level. Further, although personalized medicine can also be realized in the field of ophthalmology by genetic tests and ophthalmological agents, there is a problem of low cost-effectiveness.

The present disclosure has been made in view of such circumstances, and an object of the present disclosure is to provide a suggestion system allowing easy performing of personalized treatment tailored to a patient.

A suggestion system includes: a terminal device installed in a hospital; and a trained model connected to the terminal device and configured to assist in determining disease discovery. The terminal device includes a processor configured to: receive at least numerical data obtained by ophthalmic diagnosis and image data that is a result of image diagnosis of an eyeball; analyze a disease contained in the image data from similarity between a feature of the image data and a feature of an existing ophthalmic image sample using the trained model; and determine and suggest an optimum treatment for a patient based on the numerical data and an analysis result of the image data.

According to the suggestion system of the present disclosure, personalized treatment optimum for the patient can be performed easily.

### BRIEF DESCRIPTION OF DRAWINGS

FIG. 1 is an overall diagram of a suggestion system according to an illustrative embodiment of the present disclosure;

FIG. 2 is a diagram illustrating details of each configuration of the suggestion system;

FIG. 3 is a flowchart of an optimum treatment suggestion process performed by a terminal device;

FIG. 4 is a diagram illustrating a method of analyzing image data;

FIG. 5 is a diagram illustrating display contents of a terminal display unit (electronic medical record);

FIG. 6 is a diagram illustrating an analysis example of a fundus image and an example of suggestion information;

FIG. 7 is a diagram illustrating an analysis example of the fundus image and an example of the suggestion information (another form);

FIG. 8 is a diagram illustrating an analysis example of an OCT image and a suggestion;

FIG. 9 is an overall diagram of a suggestion system according to a modification of the present disclosure; and

FIG. 10 is a diagram illustrating details of each configuration of a suggestion system (modification).

### DETAILED DESCRIPTION

Hereinafter, an illustrative embodiment of the present disclosure will be described with reference to the drawings. The scope of the present disclosure is not limited to illustrative embodiments described here, and various modifications can be made without departing from the gist. Further, when a plurality of upper limit values and lower limit values are described for a specific parameter, any upper limit value and any lower limit value among the upper limit values and the lower limit values can be combined to obtain a suitable numerical value range.

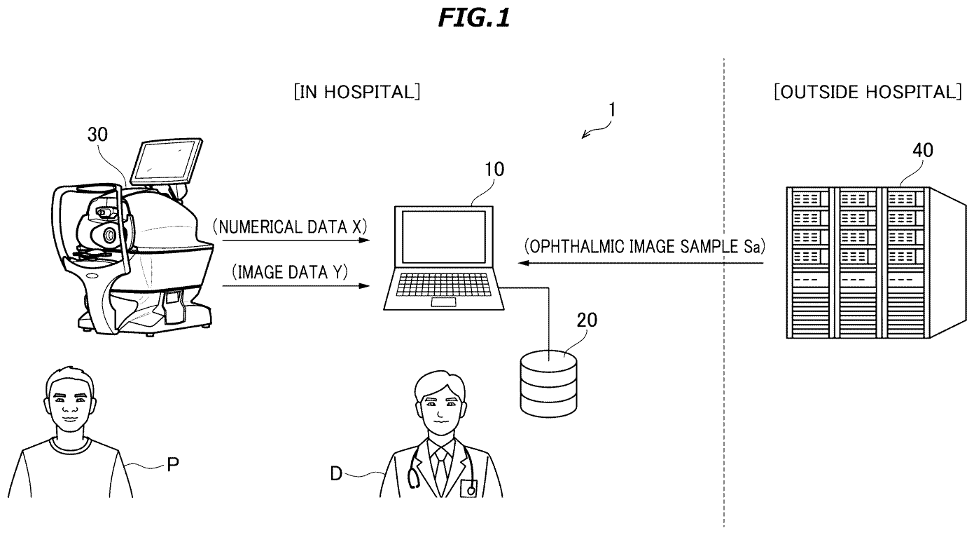

FIG. 1 is an overall diagram of a suggestion system 1 according to an illustrative embodiment of the present disclosure.

The suggestion system 1 includes a terminal device 10 mainly installed in an examination room or the like in a hospital, a trained model 20, an ophthalmic device 30, and a server 40 mainly installed outside the hospital. Since the ophthalmic device 30 is connected to the terminal device 10 via a network, a doctor D can check various data obtained in an ophthalmic diagnosis on the terminal device 10. The ophthalmic device 30 is mainly a fundus camera or an optical coherence tomography (OCT) device, but may include other devices such as a scanning laser ophthalmoscope (SLO).

The terminal device 10 is a PC, a notebook PC, a tablet terminal, or the like provided in a medical institution such as the hospital. For example, a case where the patient P undergoes an examination for glaucoma in the hospital will be described. When the patient P undergoes intraocular pressure measurement using the ophthalmic device 30, an intraocular pressure value is transmitted to the terminal device 10 as numerical data X. When an image of fundus is captured by the ophthalmic device 30, a fundus image is transmitted as image data Y to the terminal device 10. The intraocular pressure value and the fundus image are stored in the terminal device 10 and can be checked by the doctor D.

Further, since the server 40 is connected to the terminal device 10 via a network, the doctor D can constantly access an ophthalmic image sample Sa (for example, the fundus image including a symptom of the glaucoma), which is a result of an image diagnosis stored in the server 40. Since the doctor D can check recorded matters or the like in an electronic medical record in addition to a result of an ophthalmic diagnosis acquired by another medical institution, the presence or absence of an eye disease is determined in consideration of various types of information.

The doctor D determines a symptom of the glaucoma of the patient P based on at least the intraocular pressure value and the fundus image. Further, the doctor D can receive assistance in discovering a lesion or a disease by the terminal device 10 and the trained model 20.

The terminal device 10 determines a possibility of the glaucoma by comparing the fundus image of the patient P with a plurality of ophthalmic image samples Sa using the trained model 20. The glaucoma can be generally estimated based on the intraocular pressure value, but glaucoma with normal intraocular pressure also exists. The suggestion system 1 according to the present disclosure can make a quick and accurate determination without overlooking various symptoms of the glaucoma under the assistance of the trained model 20.

By the determination on the terminal device 10, for example, the presence or absence of glaucoma of the patient P and a degree of progress are displayed on a display unit. Further, suggestions for personalized treatment and medicines to be prescribed tailored to the patient P are displayed.

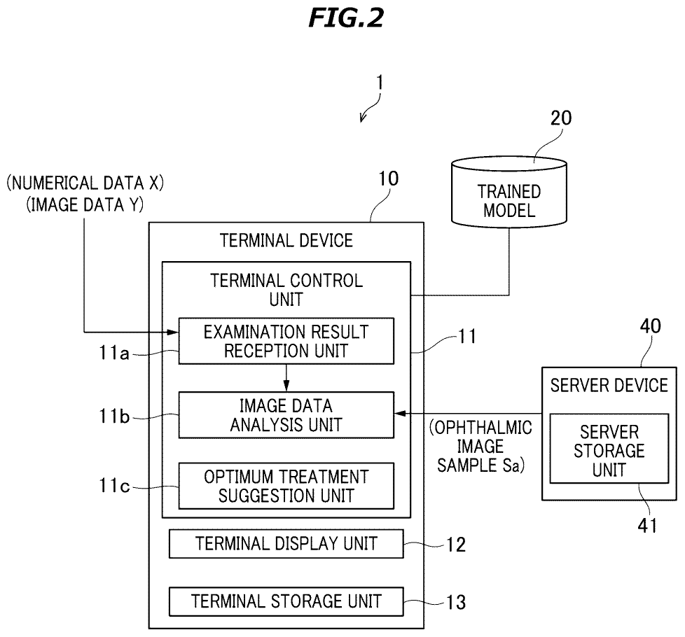

FIG. 2 is a diagram illustrating details of each configuration of the suggestion system 1. Hereinafter, internal configurations of the terminal device 10 and the server 40 and details of the trained model 20 will be described.

The terminal device 10 includes a terminal control unit 11, a terminal display unit 12, and a terminal storage unit 13 therein. Further, the terminal control unit 11 is a processor (CPU, GPU, FPGA, or the like) capable of mainly analyzing the image data Y.

The terminal control unit 11 includes an examination result reception unit 11a, an image data analysis unit 11b, and an optimum treatment suggestion unit 11c. The examination result reception unit 11a receives the numerical data X and the image data Y transmitted from the ophthalmic device 30.

The image data analysis unit 11b analyzes the image data Y while referring to the ophthalmic image sample Sa, and determines the presence or absence of an eye disease and a degree of progress (stage). At this time, the image data analysis unit 11b uses the trained model 20.

Here, the trained model 20 is a machine learning database in which machine learning is performed to determine a possibility of an eye disease by receiving a relationship between a plurality of ophthalmic image samples Sa and the eye disease as learning data. The trained model 20 receives, for example, many fundus images as learning data, adds results of glaucoma, age-related macular degeneration, and the like to obtain training data to perform machine learning. Accordingly, the image data analysis unit 11b can output information such as a degree of progress of current glaucoma and a future occurrence probability. A method for the machine learning is not particularly limited, and various methods such as unsupervised learning and deep learning can be adopted.

The trained model 20 may be connected to the server 40. In this case, a possibility of a lesion or a disease is determined by the server 40, and the result is transmitted to the terminal device 10.

The optimum treatment suggestion unit 11c determines and suggests optimum treatment for the patient P based on an analysis result of the image data analysis unit 11b while referring to the numerical data X. Contents of the suggestion include a treatment plan for an eye disease, a prescription, and a reason therefor.

The terminal display unit 12 is a display that displays a result of the examination or the image diagnosis. The doctor D can check the suggestion of the optimum treatment and the like in addition to the ophthalmic diagnosis result on the terminal display unit 12. A type of the display may be any type such as liquid crystal, plasma, or organic EL, and may be a touch panel type.

The terminal storage unit 13 is a storage medium, such as a semiconductor memory, an optical disk, or a magnetic disk into which data can be written. The ophthalmic image sample Sa of the server 40 may be downloaded by the doctor D, stored in the terminal storage unit 13, and accessed when necessary.

Next, the server 40 includes a server storage unit 41 therein. The server storage unit 41 is a storage medium, such as a semiconductor memory, an optical disk, or a magnetic disk into which data can be written. The server storage unit 41 stores previously obtained ophthalmic diagnosis results (numerical values of the examination and the ophthalmic image samples Sa). The ophthalmic image samples Sa of the server storage unit 41 are separated into groups based on eye diseases such as the glaucoma and the age-related macular degeneration, and also into groups based on the degree of progress of each eye disease.

A type of the server device 40 is not particularly limited, and may be, for example, a cloud server. In the present illustrative embodiment, the server 40 is assumed to be of a cloud type and is outside the hospital, but may be a related-art server installed in the hospital.

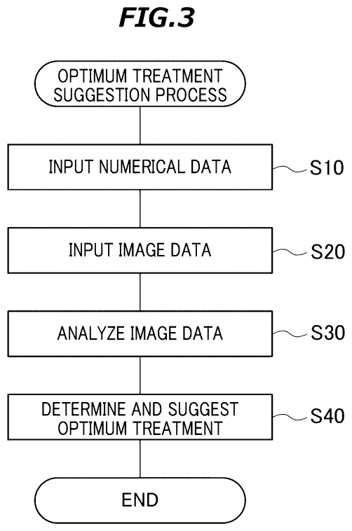

Next, with reference to FIG. 3, a flowchart of an optimum treatment suggestion process will be described. The optimum treatment suggestion process is a process performed on the terminal device 10, and is premised on the fact that the ophthalmic diagnosis is performed by the ophthalmic device 30.

First, in step S10, the numerical data X of the ophthalmic diagnosis is input to the terminal device 10. The numerical data X such as the intraocular pressure value and a field of view is received by the examination result reception unit 11a of the terminal device 10. Thereafter, the optimum treatment suggestion process proceeds to step S20.

In step S20, the image data Y of the ophthalmic diagnosis is input to the terminal device 10. The image data Y such as the fundus image and an OCT image is received by the examination result reception unit 11a. Thereafter, the optimum treatment suggestion process proceeds to step S30.

In step S30, the terminal device 10 analyzes the image data Y. Specifically, the image data analysis unit 11b of the terminal device 10 analyzes an eye disease contained in the image data Y from similarity between a feature of the image data Y and a feature of the existing ophthalmic image sample Sa using the trained model 20. Then, the image data analysis unit 11b determines the presence or absence of an eye disease read from the image data Y and a stage of the disease.

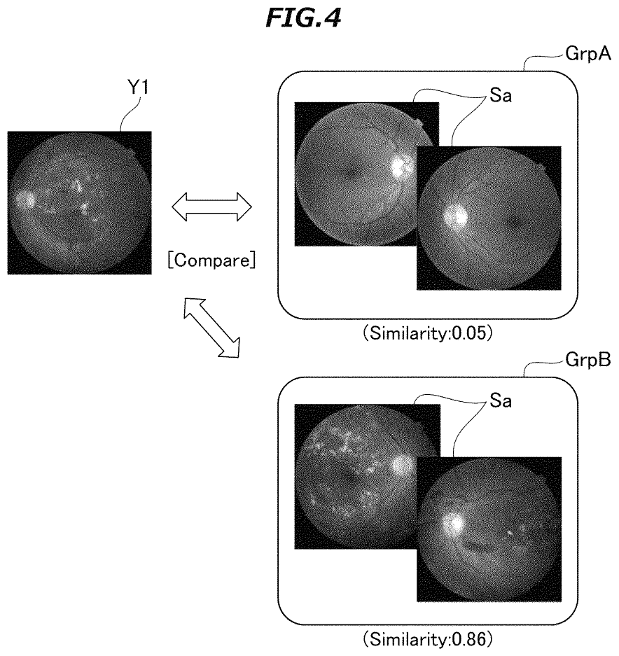

FIG. 4 is a diagram illustrating a method of analyzing the image data Y of the present step. The following analysis is internal processing of the image data analysis unit 11b and is not displayed on the terminal display unit 12 or the like.

Image data Y1 in FIG. 4 is one piece of the image data Y, and is the fundus image of the patient P obtained by the ophthalmic diagnosis. The image data analysis unit 11b extracts a feature from the image data Y1. For example, a feature specific to the presence or absence of an eye disease such as the glaucoma, the age-related macular degeneration, and diabetic retinopathy, or the stage of each eye disease is extracted from the image data Y1.

GrpA is a group in which the ophthalmic image samples Sa of glaucoma stage 1 are collected, and a feature specific to stage 1 is already extracted. Further, GrpB is a group in which the ophthalmic image samples Sa of glaucoma stage 2 are collected, and a feature specific to stage 2 is already extracted. In addition, many groups such as groups of glaucoma stages 3 and 4, groups of age-related macular degeneration stages 1 to 4, and groups of diabetic retinopathy stages 1 to 4 are prepared.

The image data analysis unit 11b sequentially compares the feature of the image data Y1 with the feature of each group, and determines a group having a highest degree of similarity. In an example of FIG. 4, similarity between the image data Y1 and GrpA is 0.05, whereas similarity between the image data Y1 and GrpB is a high numerical value of 0.86. Therefore, the image data analysis unit 11b determines that the image data Y1 is close to the feature of glaucoma stage 2 in GrpB and the possibility is high.

Returning to FIG. 3, in step S40, the terminal device 10 determines and suggests the optimum treatment. Specifically, the optimum treatment suggestion unit 11c of the terminal device 10 determines and suggests the optimum treatment for the patient P based on the numerical data X and the analysis result (step S30) of the image data Y1 from the image data analysis unit 11b. A specific example of the suggestion displayed on the terminal display unit 12 will be described later. As described above, the optimum treatment suggestion process ends.

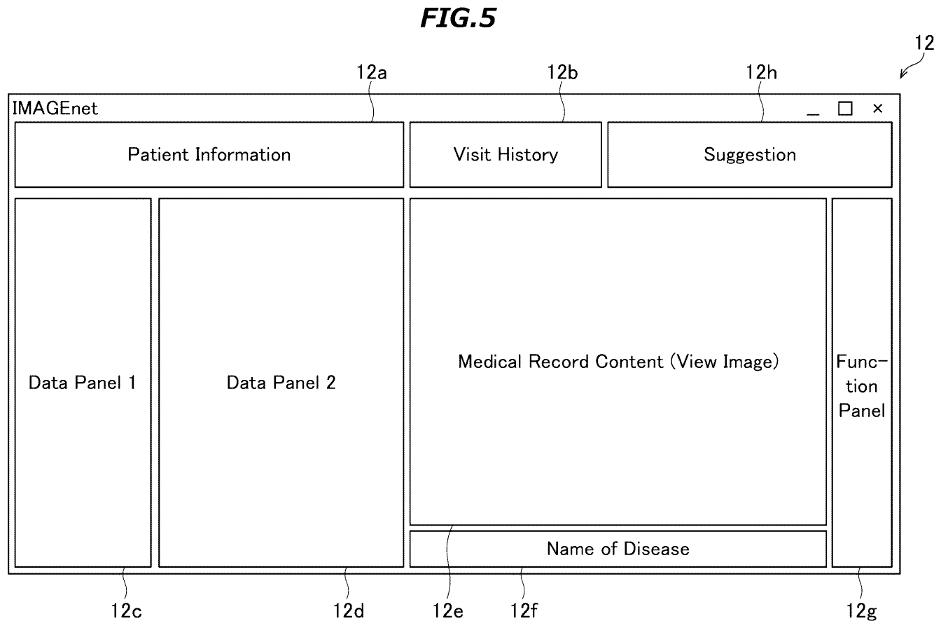

Next, a display example of the terminal device 10 (terminal display unit 12) will be described with reference to FIGS. 5 to 8.

FIG. 5 is a screen of the electronic medical record of the suggestion system 1 displayed on the terminal display unit 12. A region 12a is a region of patient information (Patient Information), and information such as the name, age, sex, and address of a patient (subject) is displayed therein. Further, a region 12b is a region of hospital visit history (Visit History), and information such as a medical institution at which the patient receives the examination, a date and time, and a medicine prescribed at that time are displayed therein.

A region 12c is a data panel (Data Panel 1) on which an examination result is displayed when a certain examination date is designated. In the region 12c, for example, information such as a visual acuity and an intraocular pressure value obtained in the examination of that day and a discovered disease are displayed. A mode in which a surgery history of the patient may be displayed.

A region 12d is also a data panel (Data Panel 2) on which an examination result is displayed, and an examination result other than that displayed in the region 12c is displayed. In the region 12d, information such as the visual acuity and the intraocular pressure value of a day different from that of the region 12c may be displayed to allow comparison, or other examination results of the same day as that of the region 12c may be displayed.

A region 12e is a region in which the recorded items (Medical Record Content) in the electronic medical record are displayed. Since the region 12e is a relatively large region, a result of the image data (Image View) can also be displayed.

A region 12f is a region in which a name of the eye disease (Name of Disease) is displayed. Further, a region 12g is a region of a function button panel (Function Panel) for switching various displays. Since it may be desired to compare a plurality of pieces of image data or progress graphs side by side, the image data or the like may be displayed in the regions 12a to 12h in a superimposed manner.

The region 12h is a region of suggestion information (Suggestion), and displays a suggestion for personalized treatment and prescription tailored to the patient P made by the optimum treatment suggestion unit 11c. In the region 12h, information on a reason for the current suggestion and a side effect may be displayed together.

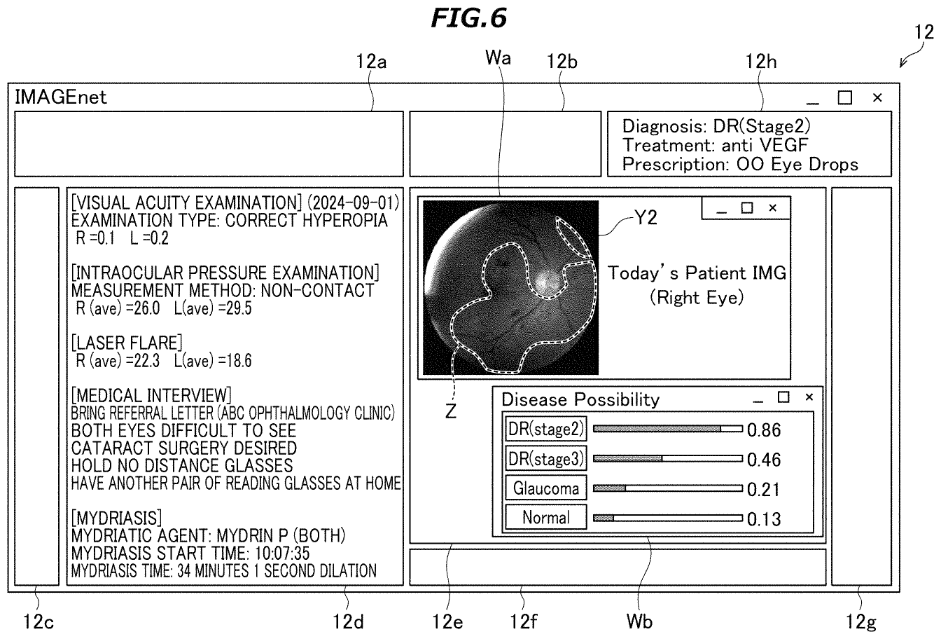

FIG. 6 is a diagram illustrating an analysis example of the fundus image and an example of the suggestion information.

First, a result of the ophthalmic diagnosis for the patient P today (2024-Sep.-01) is displayed in the region 12d of the terminal display unit 12. Specifically, a visual acuity examination, an intraocular pressure examination, laser flare, and the like (numerical data X), and a medical interview result and a mydriatic state are displayed.

Next, image data Y2 of the ophthalmic diagnosis for the patient P is displayed in the region 12e of the terminal display unit 12. In a window Wa, a fundus image (right eye) of today is displayed, and in a window Wb, an analysis result from the image data analysis unit 11b is displayed. The display indicates that it is determined, from a portion surrounded by a curve Z on the image data Y2 (fundus image), that a possibility of stage 2 of the diabetic retinopathy (DR) is highest (similarity: 0.86).

In the region 12h of the terminal display unit 12, suggestion information determined by the optimum treatment suggestion unit 11c based on the numerical data X and the image data Y2 is displayed. Specifically, in the region 12h, treatment and prescription most suitable for the patient P are suggested in addition to the diagnosis “DR (Stage 2)”. The information on a reason for selecting the treatment and prescription and a side effect may be displayed together. The doctor D refers to the suggestion in the region 12h to make a final determination about the treatment and the prescription for the patient P.

The optimum treatment suggestion unit 11c preferably determines and suggests an optimum treatment for the patient P by giving highest priority to the analysis result of the image data Y2 (fundus image in the window Wa) from the image data analysis unit 11b. This is because, when the numerical data X is prioritized, the same suggestion may be made for patients with similar numerical values (for example, intraocular pressure values). Further, in terms of the analysis of the image data Y2, a current image analysis technique is more rapid and accurate than a human being, and is not influenced by a biased feeling of the doctor in charge, which is advantageous.

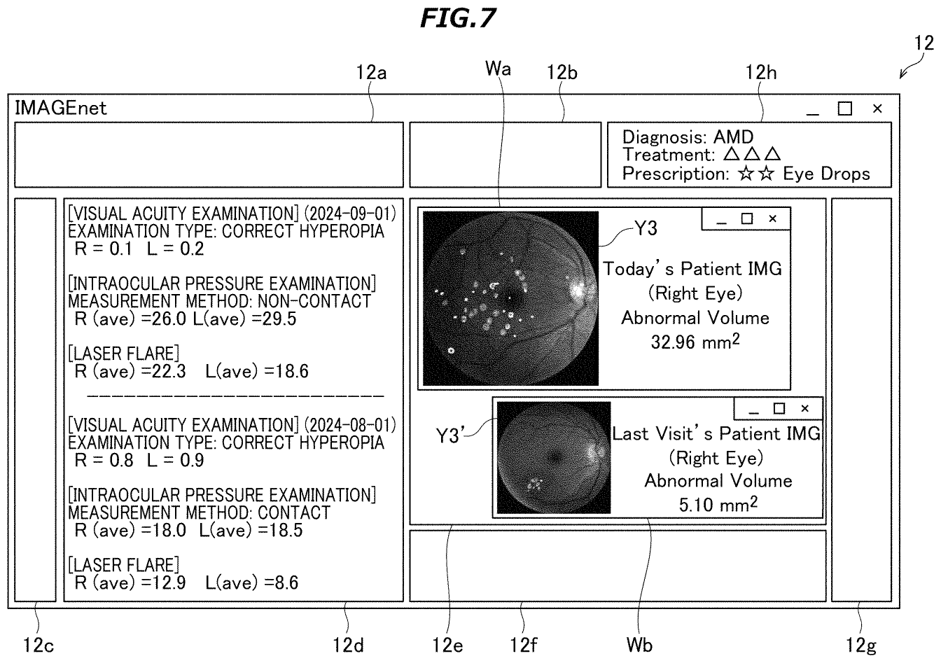

FIG. 7 is a diagram illustrating an analysis example of the fundus image and an example of the suggestion information (another form).

First, in the region 12d of the terminal display unit 12, a result of the ophthalmic diagnosis for the patient P of today (2024-Sep.-01) and a result of the ophthalmic diagnosis of a previous hospital visit date (2024-Aug.-01) are displayed. Specifically, the visual acuity examination, the intraocular pressure examination, the laser flare, and the like (numerical data X) are displayed.

Next, image data Y3 and Y3′ of the ophthalmic diagnosis for the patient P is displayed in the region 12e of the terminal display unit 12. In the window Wa, a fundus image of today (right-eye image data Y3) is displayed, and in the window Wb, a fundus image of a previous hospital visit date (right-eye image data Y3′) is displayed. In the analysis, attention is paid to an area of a specific structure (Abnormal Volume), and an area of today (image data Y3) increases to 32.96 mm² while an area of a previous examination date (image data Y3′) is 5.10 mm². The image data analysis unit 11b has a function of being able to easily calculate and quantify the area, and can obtain a result more accurate than that visually determined by the doctor P.

In the region 12h of the terminal display unit 12, suggestion information determined by the optimum treatment suggestion unit 11c based on the numerical data X and the image data Y3 is displayed. Specifically, in the region 12h, treatment and prescription most suitable for the patient P are suggested in addition to the diagnosis “AMD (age-related macular degeneration)”.

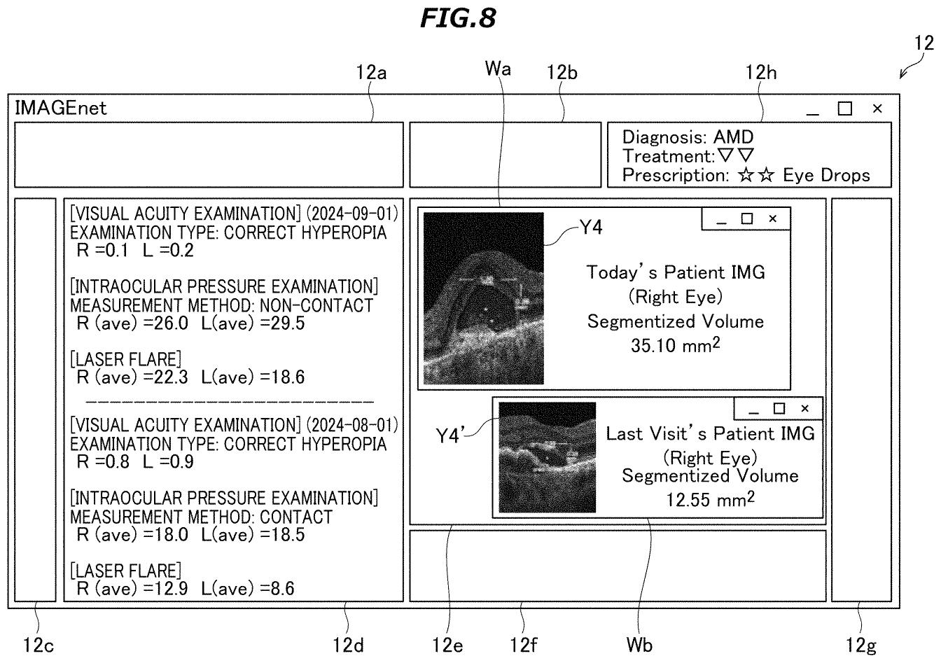

Next, an analysis example of the OCT image and an example of the suggestion information will be described with reference to FIG. 8.

First, in the region 12d of the terminal display unit 12, a result of the ophthalmic diagnosis for the patient P of today (2024-Sep.-01) and a result of the ophthalmic diagnosis of a previous hospital visit date (2024-Aug.-01) are displayed. Specifically, the visual acuity examination, the intraocular pressure examination, the laser flare, and the like (numerical data X) are displayed.

Next, image data Y4 and Y4′ of the ophthalmic diagnosis for the patient P is displayed in the region 12e of the terminal display unit 12. In the window Wa, an OCT image of today (right-eye image data Y4) is displayed, and in the window Wb, an OCT image of a previous hospital visit date (right-eye image data Y4′) is displayed. In the analysis, attention is paid to a retinal edema area as the area of the specific structure (Segmentized Volume), and an area of today (image data Y4) increases to 35.10 mm² while an area of a previous examination date (image data Y4′) is 12.55 mm². The image data analysis unit 11b has a function of being able to easily calculate and quantify the area, and can recognize a difference even if an apparent size is the same.

In the region 12h of the terminal display unit 12, suggestion information determined by the optimum treatment suggestion unit 11c based on the numerical data X and the image data Y4 is displayed. Specifically, in the region 12h, treatment and prescription most suitable for the patient P are suggested in addition to the diagnosis “AMD (age-related macular degeneration)”. In this way, the terminal device 10 of the present disclosure emphasizes the analysis of the image data Y4, receives assistance from the trained model 20, and selects the personalized treatment and prescription optimum for the patient P.

In the suggestion system 1 of the present disclosure, as described above, the terminal device 10 can easily suggest the selection of the personalized treatment and prescription optimum for the patient P. In the case of an eye disease, personalized medicine is realized by the suggestion system 1 by using the image data Y even if the patient P is not subjected to a high-cost genetic test or the like. The present disclosure is not limited to the illustrative embodiment described above and may be implemented in various modes without departing from the gist thereof. For example, the suggestion system 1 of the present disclosure can be applied to diseases other than ophthalmology requiring image diagnosis.

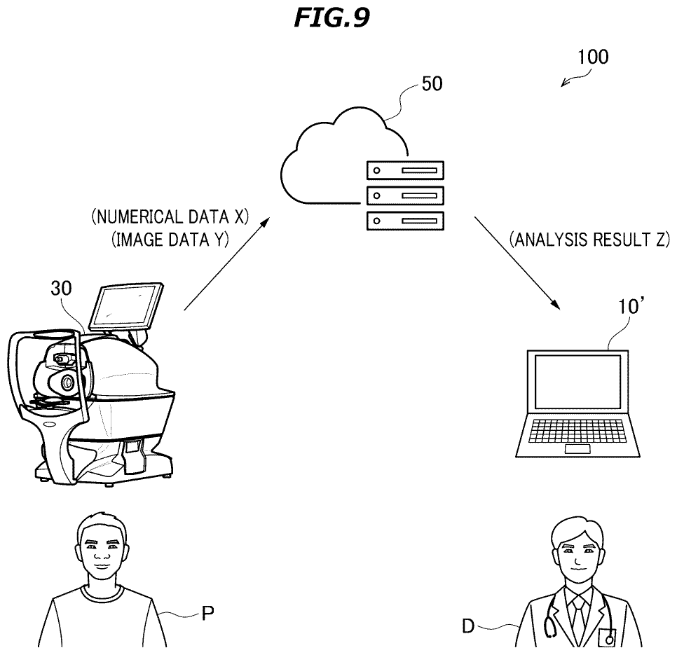

In a modification shown in FIG. 9, the suggestion system 100 is configured in such a manner that the ophthalmic device 30 and a cloud server 50 (with a trained model) are connected via a network. Further, the cloud server 50 includes an image data analysis unit, and can use the trained model.

As shown in FIG. 9, the numerical data X and the image data Y are transmitted from the ophthalmic device 30 to the cloud server 50, and a possibility of a lesion or a disease is determined in the cloud server 50. Further, a terminal device 10′ and the cloud server 50 are connected via a network, and an analysis result Z is transmitted to the terminal device 10′.

The doctor D views at least the numerical data X and the image data Y to determine a possibility of the eye disease (the glaucoma, the age-related macular degeneration, the diabetic retinopathy, or the like) of the patient P. Further, the doctor D can receive assistance in discovering an eye disease by an image analysis function (analysis result Z) of the cloud server 50.

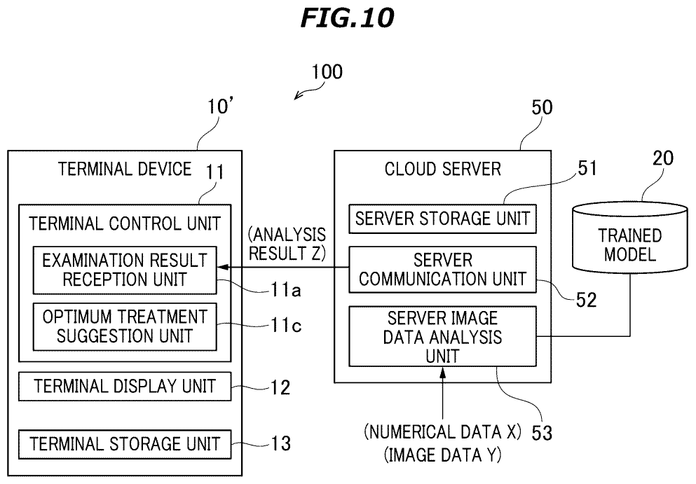

FIG. 10 is a block diagram of each configuration constituting a medical system 100 according to a modification. In the following description, the same components as those in the illustrative embodiment shown in FIG. 2 are denoted by the same reference numerals, and a description thereof may be partially omitted.

When the ophthalmic device 30 is a fundus camera, the fundus image of the patient P is captured by an imaging unit (not shown). The captured fundus image is transmitted as the image data Y to the cloud server 50 by a communication unit (not shown). The communication unit may transmit personal information (the age, the gender, or the like) of the patient together with the numerical data X and the image data Y.

The cloud server 50 includes a server storage unit 51, a server communication unit 52, and a server image data analysis unit 53 therein. In the modification, a server computer outside the hospital is assumed, but a related-art server computer installed inside or outside the hospital may be used.

The server storage unit 51 is a storage medium, such as a semiconductor memory, an optical disk, or a magnetic disk into which data can be written. The server storage unit 51 stores previously obtained ophthalmic diagnosis results (numerical values of the examination and the ophthalmic image samples Sa).

The server communication unit 52 exchanges data with the terminal device 10′ and the ophthalmic device 30. The server communication unit 52 receives the image data Y and the like transmitted from the ophthalmic device 30. Further, the server communication unit 52 transmits the analysis result Z and the like to the terminal device 10′.

The server image data analysis unit 53 analyzes the image data Y while referring to the ophthalmic image samples Sa, and estimates the eye disease and determines the degree of progress (stage). At this time, the server image data analysis unit 53 uses the trained model 20. The server image data analysis unit 53 is a processor (CPU, GPU, FPGA, or the like) that can mainly perform image analysis on image data by AI.

The terminal device 10′ includes the terminal control unit 11, the terminal display unit 12, and the terminal storage unit 13 therein. Further, the terminal control unit 11 mainly includes the examination result reception unit 11a and the optimum treatment suggestion unit 11c. The terminal control unit 11 includes, for example, a processor such as a central processing unit (CPU). The processor cooperates with a memory (including the terminal storage unit 13) in the terminal device 10′, and thus each process can be realized.

Here, the examination result reception unit 11a receives the analysis result Z transmitted from the cloud server 50. The optimum treatment suggestion unit 11c determines and suggests an optimum treatment for the patient P based on the numerical data X and the analysis result Z. Further, the terminal display unit 12 displays the suggestion information determined by the optimum treatment suggestion unit 11c.

In this way, the terminal device 10′ of the suggestion system 100 of the present disclosure can easily suggest the selection of the personalized treatment and prescription optimum for the patient P. Further, personalized medicine is realized by the suggestion system 100 by using the image data Y even if the patient P is not subjected to a high-cost genetic test or the like.

The suggestion system of the present disclosure has the following operations and effects.
(1) A suggestion system includes:
a terminal device installed in a hospital; and
a trained model connected to the terminal device and configured to assist in determining disease discovery,
wherein the terminal device includes a processor configured to:
   receive at least numerical data obtained by ophthalmic diagnosis and image data that is a result of image diagnosis of an eyeball,
   analyze a disease contained in the image data from similarity between a feature of the image data and a feature of an existing ophthalmic image sample using the trained model, and
   determine and suggest an optimum treatment for a patient based on the numerical data and an analysis result of the image data from the image data analysis unit.

In the suggestion system of the present disclosure, when the patient undergoes ophthalmic diagnosis, the processor receives at least numerical data and image data of an ophthalmic diagnosis result. The numerical data is used to grasp a disease of the patient and a symptom level.

In the analyzing, the processor refers to the current image data and ophthalmic image samples (past diagnosis results) to analyze whether the image data of the patient includes a sign of a lesion or a disease. Further, in the determining and suggesting, the processor is configured to determine and suggest optimum treatment for the patient based on the numerical data and an analysis result of the image data. In this way, the present suggestion system can easily suggest personalized treatment optimum for the patient by making a determination based on the analysis result obtained by analyzing the image data in addition to the numerical data of the patient.
(2) In the suggestion system of the present disclosure, the trained model preferably performs machine learning to determine a possibility of a disease by receiving a relationship between the ophthalmic image sample and the disease as learning data.

The trained model is connected to the terminal device, but performs machine learning to determine a possibility of the disease by receiving the relationship between the ophthalmic image sample and the disease as the learning data. Since the processor determines a possibility of a disease by comparing the image data of the patient with a plurality of ophthalmic image samples using the trained model, the presence or absence of a disease, a degree of progress, and the like can be determined objectively without bias.
(3) Further, in the suggestion system of the present disclosure, in the determining and suggesting, the processor preferably determines and suggests the optimum treatment for the patient by giving highest priority to the analysis result of the image data.

Some diseases are overlooked when the numerical data and the image data are fairly evaluated or when the numerical data is evaluated with emphasis thereon. Therefore, the processor determines and suggests the optimum treatment by giving the highest priority to the analysis result of the image data. Accordingly, the present suggestion system can prevent overlooking of a specific disease.
(4) Further, in the suggestion system of the present disclosure, it is preferable that the image data includes a fundus image, and in the analyzing, the processor is configured to quantify a specific structure in the fundus image to determine a possibility of a disease.

When the image data includes the fundus image, the processor quantifies (the number, an area, or the like), for example, a nipple shape of fundus and other specific structures to determine a possibility of a disease. Accordingly, the present suggestion system detects various diseases appearing in the fundus image.
(5) In the suggestion system of the present disclosure, it is preferable that the image data includes an optical coherence tomography (OCT) image, and in the analyzing, the processor is configured to quantify a specific structure in the OCT image to determine a possibility of a disease.

When the image data includes the OCT image, in the analyzing, the processor quantifies a specific structure contained in a layered structure of the fundus (a size of edema or the like) to determine a possibility of a disease. Accordingly, the present suggestion system detects various diseases appearing in the OCT image.

What is claimed is:

1. A suggestion system comprising:
a terminal device installed in a hospital; and
a trained model connected to the terminal device and configured to assist in determining disease discovery,
wherein the terminal device includes a processor configured to:
receive at least numerical data obtained by ophthalmic diagnosis and image data that is a result of image diagnosis of an eyeball;
analyze a disease contained in the image data from similarity between a feature of the image data and a feature of an existing ophthalmic image sample using the trained model; and
determine and suggest an optimum treatment for a patient based on the numerical data and an analysis result of the image data.

2. The suggestion system according to claim 1, wherein the trained model performs machine learning to determine a possibility of a disease by receiving a relationship between the ophthalmic image sample and the disease as learning data.

3. The suggestion system according to claim 1, wherein in the determining and suggesting, the processor is configured to determine and suggest the optimum treatment for the patient by giving highest priority to the analysis result of the image data.

4. The suggestion system according to claim 1,
wherein the image data includes a fundus image, and
wherein in the analyzing, the processor is configured to quantify a specific structure in the fundus image to determine a possibility of a disease.

5. The suggestion system according to claim 1,
wherein the image data includes an optical coherence tomography (OCT) image, and
wherein in the analyzing, the processor is configured to quantify a specific structure in the OCT image to determine a possibility of a disease.

6. A suggestion system comprising:
a terminal device installed in a hospital; and
a server including a trained model connected to the terminal device and configured to assist in determining disease discovery,
wherein the server includes a processor configured to analyze, based on image data which is obtained by ophthalmic diagnosis and is a result of image diagnosis of an eyeball, a disease contained in the image data form similarity between a feature of the image data and a feature of an existing ophthalmic image sample using the trained model, and
wherein the terminal device includes a controller configured to determine and suggest an optimum treatment for a patient based on numerical data obtained by the ophthalmic diagnosis and an analysis result of the image data from the processor of the server.
